(19) United States
(12) Patent Application Publication
(10) Pub. No.: US 2025/0345363 A1
(43) Pub. Date: Nov. 13, 2025

(54) ANTIMICROBIAL AND ANTIBIOFILM COMPOSITIONS AND USES THEREOF

(71) Applicant: MHCombiotic Inc., Calgary (CA)

(72) Inventors: Ali PORMOHAMMAD, Calgary (CA); Raymond J. TURNER, Calgary (CA)

(21) Appl. No.: 19/093,155

(22) Filed: Mar. 27, 2025

Related U.S. Application Data
(60) Provisional application No. 63/646,215, filed on May 13, 2024.

(51) Int. Cl. A61K 33/30; A61K 31/05; A61K 31/352; A61K 47/18; A61L 15/44; A61P 31/04

(52) U.S. Cl. CPC A61K 33/30; A61K 31/05; A61K 31/352; A61K 47/186; A61L 15/44; A61P 31/04; A61L 2420/06

(57) ABSTRACT

A composition having antimicrobial effects that may be used to reduce, prevent, or eliminate infection or inflammation in animals, including zinc and carvacrol. The zinc and carvacrol may be introduced in a molar ratio from about 1:1 to about 1:10, and may be mixed with various additives and excipients. The composition may be used or applied to produce anti-microbial and/or anti-inflammatory effects.

### CLAIM OF PRIORITY

The present patent application includes subject matter disclosed in and claims priority to U.S. provisional patent application Ser. No. 63/646,215 filed May 13, 2024, entitled “Antimicrobial And Antibiofilm Compositions And Uses Thereof”, incorporated herein by reference, and which describe an invention made by the present inventors.

### FIELD

This specification relates generally to antimicrobial and antibiofilm compositions and their uses, including but not limited to synergistic antimicrobial and antibiofilm compositions and their prevention and treatment of infections.

### BACKGROUND

The following paragraphs are not an admission that anything discussed in them is prior art or part of the knowledge of persons skilled in the art.

Silver is a common component of topical treatments of local infection, such as infection of burns and wounds.

However, widespread use of silver-based treatments may lead to an increase in antimicrobial resistance (AMR) of microbes such as bacteria, viruses and fungi. For example, overuse of silver-containing compounds may result in plasmid-mediated silver resistance in certain bacteria. Thus, the efficacy of silver-based treatments of local infection may be reduced overall.

### SUMMARY OF THE INVENTION

The following introduction is intended to introduce the reader to this specification but not to define any invention. One or more inventions may reside in a combination or sub-combination of the composition components, uses, apparatus elements or method steps described below or in other parts of this document. The inventors do not waive or disclaim their rights to any invention or inventions disclosed in this specification merely by not describing such other invention or inventions in the claims.

Non-silver-based approaches in antimicrobial treatments of topical infection, such as in burn or wound care, may be beneficial for antimicrobial applications.

In one aspect, the present disclosure provides a composition comprising zinc and carvacrol in a molar ratio from about 1:about 0.5 to about 1:about 10, such as a ratio of about 1:about 3.

In another aspect, the present disclosure provides a composition comprising an antimicrobially synergistic combination of zinc, carvacrol, delta-9-tetrahydrocannabinol (THC), cannabidiol (CBD), and resveratrol (such as C₁₄H₁₂O₃). In the context of the present disclosure, the term “antimicrobially synergistic combination” refers to a combination of components whose antimicrobial effects are at least two times greater than that of each component alone as measured by the synergism combination antibacterial percent efficacy as well as fractional inhibitory concentration (FIC) or the fractional bactericidal concentration (FBC), as applicable.

In yet another aspect, the present disclosure provides a composition comprising: zinc, carvacrol, THC, CBD, resveratrol, and at least one surfactant: an ionic or anionic surfactant, such as a chelating/complexing agent, an anionic surfactant, a cationic surfactant, and/or a nonionic surfactant. Optionally, the composition may further comprise one or more preservative, for example, hydrogen peroxide H₂O₂, polyhexamethylene biguanide PHMB, or other preservatives.

In yet another aspect, the present disclosure provides a composition comprising: zinc sulfate, carvacrol, carboxymethyl cellulose (CMC), carbomer, D-panthenol, and a copper peptide.

In yet another aspect, the present disclosure provides a composition comprising: zinc sulfate, carvacrol, sodium chloride, and cocamidopropyl betaine.

In yet another aspect, the present disclosure provides a method of making a composition comprising: combining zinc and carvacrol in a molar ratio from about 1:about 0.5 to about 1:about 10, such as a ratio of about 1:about 3.

In yet another aspect, the present disclosure provides a method of making a composition comprising: combining zinc, carvacrol, THC, and CBD in a molar ratio from about 1:about 3:about 0.1:about 0.1.

In yet another aspect, the present disclosure provides a method of making a composition comprising: combining zinc, carvacrol, THC, CBD and resveratrol in a molar ratio from about 1:about 3:about 0.1:about 0.1:about 0.1.

Various additives may be added, such as surfactants, buffers, carriers, preservatives, solvents, and thickeners, antimicrobials, antibiotics, stabilizing agents, herbs, vitamins, minerals, extracellular matrix, pH adjusters (for example, sodium hydroxide and/or other alkaline substances, but also hydrochloride, sodium acid pyrophosphate, acetate, etc.), peptides, and/or proteins. The composition, or application thereof, may be made free of silver.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a graph of disruption potency of antimicrobial compositions of biofilm biomass of Staphylococcus aureus (S. aureus) after about 30 minutes of exposure.

FIG. 2 is a graph of disruption potency of antimicrobial compositions of biofilm biomass of Pseudomonas aeruginosa (P. aeruginosa) after about 30 minutes of exposure.

FIG. 3 is a graph of disruption potency of antimicrobial compositions of biofilm biomass of mixed S. aureus, P. aeruginosa and Candida albicans (C. albicans) after about 30 minutes of exposure.

FIG. 4 is a graph of eradication potency of antimicrobial compositions of biofilm biomass of Staphylococcus aureus (S. aureus) after 5 minutes (5 min) exposure.

FIG. 5 is a graph of eradication potency of antimicrobial compositions of biofilm biomass of Pseudomonas aeruginosa (P. aeruginosa) after 5 min exposure.

FIG. 6 is a graph of eradication potency of antimicrobial compositions of biofilm biomass of mixed S. aureus, P. aeruginosa and Candida albicans (C. albicans) after 5 min exposure.

FIG. 7 illustrates a heat map illustrating antibacterial efficacy of bandages treated with a composition according to the present disclosure.

FIG. 8 illustrates a heat map illustrating comparative antibacterial efficacy of bandages treated with a composition according to the present disclosure.

FIG. 9 illustrates histological effects such as inflammatory cells scattering of the control and test article's injection sites.

### DETAILED DESCRIPTION

### Antimicrobial Compositions Comprising Zinc and Carvacrol

Compositions comprising both zinc a carvacrol may have particular antimicrobial effects that may be used to reduce, prevent, or eliminate infection in animals including humans.

In the context of the present disclosure, the term “zinc” refers to any salt, molecule, compound, or substance comprising the element zinc including, but not limited to: zinc nitrate, zinc sulfate, zinc chloride, zinc oxide, zinc chelates, zinc complexes and nanomaterials comprising the element zinc or combinations thereof.

In the context of the present disclosure, the term “carvacrol” refers to 5-Isopropyl-2-methylphenol, but may also refer to analogous structures such as liquid C₁₀H₁₄O and/or Isopropyl-o-cresol, 2-Hydroxy-p-cymene, 2-Methyl-5-isopropylphenol, etc. Carvacrol is known as a natural monoterpene phenol derivative of cymene. Carvacrol is a member of phenols, a p-menthane monoterpenoid and a known botanical anti-fungal agent, and may derive from a hydride of a p-cymene. Carvacrol may be isolated from natural sources or synthetic product, either source being useful for the present invention. Carvacrol, also known as cymophenol, C₆H₃(CH₃)(OH)C₃H₇, is a monoterpenoid phenol, and can be in the form of a thick, oily substance, (CH₃)₂CHC₆H₃(CH₃) (OH), as extracted from various essential oils.

In the context of the present disclosure, the term “antimicrobial” refers to a method or agent that can be against both bacteria (Gram-negative and Gram-positive) and fungi, either cradicating or preventing growth. In another term a component that may slow, inhibit, or kill a microbe. An antimicrobial component may be referred to as an antibacterial component when the microbe is bacteria.

In the context of the present disclosure, the term “antibacterial” refers to a method or agent that can be used against bacteria in either planktonic or biofilm form, either eradicating or preventing the bacteria as measured by minimum bactericidal concentration (MBC) or minimum bacteriostatic concentration (MIC). For example, a method or agent that may have bactericidal or bacteriostatic effects when a microbe is a bacteria. In another example, the component may reduce or prevent reproduction and proliferation when a microbe is a bacteria.

In the context of the present disclosure, the term “antifungal” refers to a method or agent that can be against fungi in either planktonic or biofilm form, either cradicating or preventing the fungi's growth. The method or agent may have fungicidal or fungistatic effects when the microbe is a fungus. For fungicidal effects, a minimum fungicidal concentration (MFC) was calculated, and for fungistatic effects, the minimum fungistatic concentration (MFSC) was calculated.

Such compositions may have properties such as a lower minimum inhibitory concentration (MBIC), and lower minimum bactericidal concentration (MBC) than silver-based compositions, or zinc-based compositions alone.

Various isotopes, different commercial brands, delivery formats, and structures such as nano forms of the selected agents, such as zinc, were evaluated for antimicrobial properties and any associated adverse effects. For example, both zinc sulfate and zinc oxide (in nano form) exhibited similar antimicrobial effects.

The source of zinc in the disclosed compositions may be selected according to the desired properties of the zinc source or the desired properties of the final composition such as solubility. For example, while zinc sulfate heptahydrate demonstrated antimicrobial effects comparable to zinc sulfate monohydrate, the former exhibited higher solubility with other components and therefore may be selected as a component in a composition where higher solubility is desirable.

Compositions comprising both zinc and carvacrol may demonstrate improved antimicrobial effects when combined with certain other antimicrobials such as antibiotics.

Compositions comprising both zinc and carvacrol may demonstrate improved anti-biofilm effects when combined with certain natural surfactants such as betaine-based surfactants.

### Hydrogel Compositions

Antimicrobial compositions as disclosed herein may be combined with certain additives to form a hydrogel composition, for example, for use in the topical treatment of local infection. In the context of the present disclosure, the term “additive” refers to any component that may form part of a composition, except for zinc and carvacrol; and the term “local infection” refers to an infection not in the bloodstream of an animal.

Such hydrogel compositions may be used to directly contact a local infection for a period of time for delivering the antimicrobial composition therein to the infection site. Hydrogel compositions may in addition, or alternatively, be incorporated into a dressing, which may then be applied to the site of a local infection.

A hydrogel may be used in the treatment or prevention of an acute or chronic local infection where, particular adhesion properties, moisture retention properties, degradation properties and gas permeability properties are desirable. For example, a hydrogel composition may be used for surgical incisions, superficial epidermal trauma, scalds, ulcerative wounds, burns, pressure ulcers, diabetic ulcers, or ulcers caused by radiotherapy or chemotherapy.

Additionally, a hydrogel may be used in the treatment or prevention of an acute or chronic infection where particular adhesion properties, moisture optimizing properties, degradation properties, and/or gas permeability properties are desirable. Further desirable properties may include enhanced wound healing properties, cosmetic benefits such as anti-aging and anti-acne effects, suitability for coating on medical devices, spray application, facilitation of blood hemostasis and thrombosis, promotion of tissue regeneration, and scar treatment. Other desirable properties may relate to biocompatibility, biodegradability, non-toxicity, and the ability to release therapeutic agents in a controlled manner.

In some examples the composition may be incorporated into a hydrogel comprising zinc, carvacrol, THC, CBD and resveratrol wherein the zinc, carvacrol, THC, CBD and resveratrol are present in a molar ratio from about 1:about 10:about 0.5:about 0.5:about 2to about 1:about 1:about 0.025:about 0.025:about 0.2.

A hydrogel may further comprise additives such as one or more surfactants, buffers, carriers, preservatives, solvents, or thickeners. For example, the hydrogel may comprise: zinc sulfate, carvacrol, carboxymethyl cellulose (CMC), carbomer, D-panthenol, copper peptide.

### Ointment Compositions

Antimicrobial compositions as disclosed herein may be combined with certain additives to form an ointment composition, for example, for use in the topical treatment of local infection.

Such ointment compositions may be used to directly contact a local infection for a period of time for delivering the antimicrobial composition therein to the infection site.

An ointment may be used in the treatment or prevention of an acute or chronic local infection where, particular occlusive, lower gas permeability, or anti-irritant properties are desirable. For example, an ointment composition may be used for irritated, unwounded skin.

In some examples the composition may be incorporated into an ointment comprising zinc, carvacrol, THC, CBD and resveratrol wherein the zinc, carvacrol, THC, CBD and resveratrol are present in a molar ratio from about 1:about 10:about 0.5:about 0.5:about 2to about 1:about 1:about 0.025:about 0.025:about 0.2.

An ointment may further comprise additives such as one or more surfactants, buffers, carriers, preservatives, solvents, or thickeners. For example, emulsifier wax, cetyl alcohol, glycerin, olive oil, grapeseed oil, glycerin, xanthan gum, and benzoyl peroxide or salicylic acid in anti-acne applications.

### Solution and Spray Compositions

Antimicrobial compositions as disclosed herein may be combined with certain additives to form a solution or a spray composition, for example, for use in the topical treatment of infection, dipping solutions, cleansers, serums, moisturizers, face washes, body washes, nasal washes, injections, intramuscular injectable solutions, intravenous injectable solutions, nebulizers, aerosols, vaporizers, wound irrigators, oral hygiene washes, and post-surgical washes.

Such solution or spray compositions may be used to disrupt biofilm formation or directly contact an infection for a period of time for delivering the antimicrobial composition therein to the infection site where application through rubbing or friction is undesirable.

A solution or spray may be used in the treatment or prevention of an acute or chronic local infection where, antiseptic or cleansing properties are desirable. For example, an ointment composition may be used for cuts, abrasions, blisters, and burns.

In some examples the composition may be incorporated into a spray, such as a wash or wound spray, comprising zinc, carvacrol, THC, CBD and resveratrol wherein the zinc, carvacrol, THC, CBD and resveratrol are present in a molar ratio from about 1:about 10:about 0.5:about 0.5:about 2 to about 1:about 1:about 0.025:about 0.025:about 0.2.

A solution or spray composition may further comprise additives such as one or more surfactants, buffers, carriers, preservatives, solvents, or thickeners. For example, zinc sulfate, carvacrol, sodium chloride, and cocamidopropyl betaine.

### Additives

Antimicrobial compositions as described herein may be combined with additives to form various formulations that may be suitable for particular applications. Additives may be selected according to the desired properties for a particular formulation. Examples of additives are described above, and also include surfactants, buffers, carriers, preservatives, solvents, and thickeners, antimicrobials, antibiotics, stabilizing agents, herbs, vitamins, minerals, extracellular matrix, pH adjusters (for example, sodium hydroxide and/or other alkaline substances, but also hydrochloride, sodium acid pyrophosphate, acetate, etc.), peptides, and proteins, all as are known in the art.

### Surfactants

In some examples, a hydrogel, ointment, solution or spray composition may comprise one or more ionic or nonionic surfactants, such as but not limited to: cocamidopropyl betaine, betaine, betaine-based surfactants N-lauroylsarcosince, benzalkonium chloride (BZK) in liquid or powder form, coco-glucoside (coco glucose), Tween® 20, sodium dodecyl sulfate (SDS), cetyltriromide (CTAB), ethylenediaminetetraacetic acid (EDTA), saponin, sucrose fatty acids, or sodium cocoyl isethionate (SCI), sodium lauryl sulfate, polysorbate 80, sodium lauryl sulfate, sodium laureth sulfate, stearic acid, lauric acid, sorbitan monolaurate, poloaxmer 407, polyethylene glycol (PEG), glycol monostearate, and ethoxylated fatty alcohols. Surfactants may be in liquid or powder form, providing a versatile range of options for formulation.

### Buffers

In some examples, a hydrogel, ointment, solution or spray composition may comprise one or more buffers, such as but not limited to: saline (0.9% NaCl), phosphate buffer, tris buffer, citrate buffer, acetate buffer, borate buffer, glycine buffer, HEPES buffer, MES buffer, phosphate buffered saline (PBS), or tris-borate-EDTA (TBE buffer).

### Carriers

In some examples, a hydrogel, ointment, solution or spray composition may comprise one or more carriers, such as but not limited to: saline (0.9 NaCl), phosphate buffer, tris buffer, citrate buffer, acetate buffer, borate buffer, glycine buffer, HEPES buffer, MES buffer, phosphate buffered saline (PBS), or tris-borate-EDTA (TBE buffer).

### Preservatives

In some examples, a hydrogel, ointment, solution or spray composition may comprise one or more preservatives, such as but not limited to: polyhexamethylene biguanide (PHMB), benzalkonium chloride (BZK), hydrogen peroxide (H₂O₂), hyaluronic acid, hypochlorous acid (HOCl), benzyl alcohol, methylparaben, ethylparaben, propylparaben, chlorhexidine gluconate, triclosan, phenoxyethanol, sodium benzoate, potassium sorbate, or sorbic acid.

### Solvents

In some examples, a hydrogel, ointment, solution or spray composition may comprise one or more solvents, such as but not limited to: ethanol, isopropyl alcohol, propylene glycol, glycerol, polyethylene glycol (PEG), dimethyl sulfoxide (DMSO), acetone ethyl acetate, or methanol butanol.

### Thickeners

In some examples, a hydrogel, ointment, solution or spray composition may comprise one or more thickeners, such as but not limited to: glycerin, xanthan gum, emulsifier wax, cetyl alcohol, carboxymethyl cellulose (CMC), carbomer, polyethylene glycol (PEG) 2000, PEG 3350, PVA, Carbopol 980NF, PEG 400, polydimethylsiloxane (PDMS), polyvinyl alcohol (PVA), PEG 8000, poly (p-phenylene vinylene) (PPV, or polyphenylene vinylene), D-panthenol, hydroxyethyl cellulose (HEC), hydroxypropyl methylcellulose (HPMC), sodium alginate, polyacrylamide, polyvinylpyrrolidone (PVP), polyvinylpyrrolidone-vinyl acetate copolymer (PVP-VA), poloxamer, acrylic acid polymers, polyacrylic acid, polysorbate, polyethylene oxide, guar gum, sodium carboxymethylcellulose (NaCMC), polyvinylpyrrolidone-vinyl acetate copolymer (PVP-VA), or polyethyleneimine.

### Antimicrobials

In some examples, a hydrogel, ointment, solution, or spray composition may comprise one or more other antimicrobials such as antibiotics, antimicrobial peptides, antimicrobial phages, benzalkonium chloride (BZK), polyhexamethylene biguanide (PHMB), hydrogen peroxide (H2O2), hyaluronic acid, and/or hypochlorous acid (HOCI).

### Vitamins and Minerals

In some examples, a hydrogel, ointment, solution or spray composition may comprise one or more vitamins and/or minerals, such as but not limited to: D-panthenol, vitamin E, multivitamin, vitamin C, vitamin A, vitamin B complex (including B1, B2, B3, B5, B6, B7, B9, B12), vitamin K, calcium, magnesium, zinc, iron, selenium, copper, manganese, potassium, and iodine.

### Tissue Repair or Growth Factors

In some examples, a hydrogel, ointment, or solution or spray composition may comprise one or more tissue repair, growth factors, and wound healer agents, such as but not limited to: copper peptide, epidermal growth factor (EGF), fibroblast growth factor (FGF), platelet-derived growth factor (PDGF), transforming growth factor beta (TGF-B), insulin-like growth factor (IGF), vascular endothelial growth factor (VEGF), keratinocyte growth factor (KGF), hyaluronic acid, collagen, elastin, fibronectin, and allantoin.

### Uses of Antimicrobial and Antibiofilm Compositions

Compositions according to the present disclosure, such as hydrogel, ointment, solution, or spray compositions may be used in a number of applications and formulations including but not limited to: cream, ointment, gel, hydrogel, wound dressing, solution, spray, cleanser, serum, toner, moisturizer, different types of oil, soap, toothpaste, detergent, coating different wound dressings, bandages, wetted wipes, dipping solution, wound scrapers, face/body wash, shampoo, additive/supplementary in animal foods, treatment of infections of cuts, bruises, surgical sites, lacerations, abrasions, punctures, incisions, gunshots, burns, pyoderma, otitis media, otitis externa/interna, cow udder (mastitis), respiratory/systemic, atopic dermatitis, eczema, pressure ulcers, venous and artery, leg ulcers, and diabetic foot ulcers, intramuscular/intravenous injectable solution, atomizer, sprayer, nebulizer, acrosol, vaporizer, sprinkler, dispenser, pulverizer, sprinkler, acrosol, tablets, capsules, liquids, drops, inhalers, injections, toothbrushes, toothpaste, dental floss, dental picks, sticks, oral irrigators/hygiene, wound irrigators/hygiene, nasal wash, teeth whiteners, antibiofilm products, tongue scrapers, mouthwash, animal infection control, preservative in different industries such as food, cosmetics, supplementary, and pharmaceutical products, tissue repair, anti-aging, and/or anticavity, coatings for medical stents, masks, orthopedic devices, artificial joints, tongue depressors, textiles (e.g., clothing, dressings, personal protective clothing, bedpans), orthopedic, implants, needles, bone implants, breast pumps, medical gloves, personal protective equipment, surgical equipment, sanitary towels, contact lenses, contact lenses solution, surfaces as found in hospital or other health care facilities, high-touch surfaces, care setting, restaurant, food processing facility, dairy containers and processing, dairy/food storage, dairy/food product distribution system/pipeline, slaughterhouse, laboratory, vehicle, boat, manufacture, industrial cooling system (e.g., a cooling tower), petroleum product storage, or a petroleum product distribution system/pipeline, furniture surfaces, partition curtains, handrails, doors, doorknobs, walls, floors, elevators, air vents, air filters, pipes, vessels, kitchen items, clothing, and electronic devices.

While the specific lists of additives, including excipients, are listed above and characterized as additives, surfactants, buffers, carriers, preservatives, solvents, thickeners, antimicrobials, antibiotics, stabilizing agents, herbs, vitamins, minerals, extracellular matrix, growth factors, pH adjusters, peptides, and proteins, etc. The lists are not meant to be exhaustive, but can be read to include similar compounds and additives with similar structure, composition, and/or known properties and/or reactions.

### EXAMPLES

To gain a better understanding of the invention described herein, the following examples are set forth. It should be understood that these examples are for illustrative purposes only. Therefore, they should not limit the scope of this invention in any way. In this disclosure, when using the term “about,” it is most preferably thought of as achieving the goal time or ratio within 5% of the target, or at least within 10% of the target time or ratio. For instance, about 30 minutes may be understood as 28 min 30 sec to 31 minutes thirty seconds, or at least within the range of 27 minutes to 33 minutes. Similarly with ratios, when discussing a 1:10 ratio is preferably within a 2:19 to 2:21 range, and at least a 1:9 to 1:11 range, etc., and about 1:about 10 may preferably refer within a 19:210 to 21:190, or at least within 11:90 to 9:120.

### Example 1

### Method

Samples of 11 different gram-positive and gram-negative bacteria as well as 7 different fungi, in total 18 microbes included Pseudomonas aeruginosa ATCC 27853, Pseudomonas aeruginosa PAO1, Klebsiella pneumoniae ATCC 11296, Staphylococcus aureus ATCC 25923, Methicillin resistant S. aureus (MRSA) ATCC 33591, Escherichia coli ATCC 25922,Enterococcus faecium ATCC 19434, Staphylococcus epidermidis ATCC 12228, Enterococcus faecalis ATCC 29212, Proteus mirabilisATCC 35659, Acinetobacter baumannii CDC strain AR Bank #0033, Candida albicans ATCC 90028, Candida glabrata ATCC 2001, Candida parasilosis ATCC 22019, Candida krusei ATCC 14243, Candida tropicalis ATCC 13803, Candida auris ATCC MYA-5002, Aspergillus fumigatusATCC 16404 were used.

Metal (loid)-based antimicrobials (MBAs) including, silver nitrate (AgNO3, Sigma-Aldrich, St Louis, MO, USA Lot #M3259), potassium tellurite (K2TeO3, Sigma-Aldrich, St Louis, MO, USA, Cat #P2358-12C), hydrogen tetrachloroaurate (III) trihydrate (AuCl4.3H2O, Alfa Acsar Tweksbury, MA, USA, Lot #U17G046), zinc oxide nanoparticles, <100 nm (Sigma Aldrich), zinc sulfate (ZnSO4·1H2O), and zinc sulfate (ZnSO4.7H2O) were received from Fisher Scientific (Fair Lawn, NJ, USA, Lot #723689). Plant-based components (PBCs) including Cannabidiol (CBD) (SIGMA, MO, USA, LOT #SLCC9048), (-)-11-nor-9-carboxy-Δ9-tetrahydrocannabinol (THC) (SIGMA, MO, USA, LOT #FE05081905), Cinnamaldehyde (Fisher Scientific, Ottawa, Canada, LOT #5018R23W, >98%), Tea Tree Oil (Newconatural, Australia, LOT 80038607, >98%), Nerolidol (TCI, Portland, USA LOT #IKUMH-BE, >97%), carvacrol (TCI, Portland, USA LOT #NDFYD-GT, >98%), 0-coumaric acid (SIGMA, MO, Germany, LOT #106H0966), Thymol (Fisher Scientific, Ottawa, Canada, LOT #W12E020, >98%), Resveratrol (TCI, Portland, USA LOT #QWDXI-RF, >99%), and Canada Balsam (SIGMA, MO, USA, LOT #125H2518) were used. Stock solutions of all agents were prepared at concentrations up to 500 mg/ml, while working solutions of each agent were prepared at concentrations ranging from 10 to 100 mg/ml in distilled and deionized (dd) H₂O. The stock dilutions were stored in glass vials at room temperature in a dark place for no longer than two weeks. Working solutions were freshly prepared no more than 30 minutes before experimental use by diluting the stock solutions in equal amounts of media.

Other material including polyethylene glycol (PEG) 2000 (Aldrich Chemical Co., Inc), Polydimethylsiloxane (PDMS) (Thermo Science), polyvinyl alcohol (PVA) (Thermo Science), PEG 8000, Benzalkonium chloride (BAK), Poly (p-phenylene vinylene) (PPV, or polyphenylene vinylenc), Coco Glucose (making cosmetics, WWA), Tween 20, Sodium Dodecyl Sulfate (SDS), Cetyltrimethylammonium Bromide (CTAB), Betaine (Sigma), Ethylenediaminetetraacetic Acid (EDTA), Sucrose fatty acid (TCI America), Lauroylsarcosinc (Voyageur, BC, Canada), Betaine Surfactant (Voyageur, BC, Canada), Sodium Cocoyl Isethionate-SCI (Voyageur, BC, Canada), Polyhexanide (polyhexamethylene biguanide, PHMB), Hyaluronic acid, and/or Hypochlorous acid (HOCL). Carbomer 940 USP grade (Talsen Chemicals, NY, USA) Betaine (Thermo Science lot #10243221), glycerin (Sport research, SP, CA), Copper peptide (Frostproof USA), D-Pantheno (Thermo Science, China), Saponin (T Thermo Science, Germany), Carboxy (VWR, TCI America), lodine 10% Topical USP (iodine, USA). Prontosan (USA).

Antimicrobial assays for the planktonic form of bacteria were conducted in a 96-well plate, while assays for the biofilm form were conducted using the Calgary Biofilm Device (CalBD; commercially available as the Minimum Biofilm Eradication Concentration Assay® (MBEC Assay®) [Innovotech Inc., Edmonton, Alberta, Canada]), as originally described by Ceri et al. Antimicrobial susceptibility testing of single components and compositions against the planktonic form of bacteria was performed using the MIC and MBC methods, whereas for the biofilm form, MBIC and MBEC methods were employed (18).

Mueller-Hinton Broth (MHB, BD Bacto, Oxoid, Basingstoke, UK Cat #X125A) was utilized as the growth medium and 1.0×10⁶ CFU/ml bacterial/fungi suspension used for each test.

Planktonic Susceptibility. Testing of planktonic susceptibility was conducted using MIC and MBC assessments on bacterial strains using 96-well microtiter plates. To prepare the wells for bacteria, serial dilutions of the antibacterial agents were Carried out along the rows of two microtiter plates. The antibacterials were 2-fold diluted with Mueller-Hinton broth (MHB) in each column, reaching a volume of 75 μL. The 1.0 McFarland standard was diluted 15-fold in MHB, and 75 μL of inoculum was added to the wells treated with antibacterials, resulting in a total volume of 150 μL. The microtiter plates were covered, then shake-incubated overnight at 150 rpm and 37° C. MIC was determined based on visible bacterial growth. In cases of Antibacterial opacity or ambiguous results, streaking of well cultures on Mueller-Hinton agar (MHA), overnight incubation, and subsequent comparison to controls were performed. MBC was determined by transferring 3 u L of culture from all MIC microtiter plate wells to 147 μL of MHB in fresh plates. These plates were shake-incubated overnight at 150 rpm and 37° C., and bacterial growth was visually inspected. Combination Antimicrobial Efficacy Testing. This checkerboard (96-well microtiter plates) included one column just media without bacteria/fungi and MBAs/PBCs (as a negative control), and one column without MBA/PBCs, with media, and bacteria/fungi (as a growth and positive controls control), MBAs and/or PBCs were diluted ½ across the rows (19, 20). For each analysis, the same MIC and MBC/MFC steps indicated above were conducted for surveying the bacteriostatic, bactericidal, and reading synergism effects of MBAs with PBCs combinations.

### Analysis of Combination Antibacterial % Efficacy in Comparison to Silver

Use the following formula to calculate the % Efficacy of the data in comparison to silver:

$$\% Efficacy = ( \frac{1}{M B I C * ( \frac{1}{M B I {C}_{\text{?}}} )} ) \times 100 \%$$

$$\% Efficacy = ( \frac{1}{M I C * ( \frac{1}{M I {C}_{\text{?}}} )} ) \times 100 \%$$

$$\% Efficacy = ( \frac{1}{M B C * ( \frac{1}{M B {C}_{\text{?}}} )} ) \times 100 \%$$

$$\text{?} \text{indicates text missing or illegible when filed}$$

After calculating % Efficacy for every value individually, the % Efficacy values for the combinations were averaged:

$$Average \% Efficacy for M I {C}_{\text{?}} = \frac{\% Efficacy M I {C}_{\text{?}} + \% Efficacy M I {C}_{\text{?}} + \% Efficacy M I {C}_{\text{?}}}{3} = \frac{0.11 \% + 300. \% + 976.75 \%}{3} = 425.62 \%$$

$$\text{?} \text{indicates text missing or illegible when filed}$$

These values were then again averaged throughout all bacteria (but within MIC, MBC/MFC, and MBIC categories).

$$Average \% Efficacy for M I {C}_{\text{?}} = ( Average \% Efficacy for M I {C}_{\text{?}} + Average \% Efficacy for M I {C}_{\text{?}} + Average \% Efficacy for M I {C}_{\text{?}} + Average \% Efficacy for M I {C}_{\text{?}} + Average \% Efficacy for M I {C}_{\text{?}} + Average \% Efficacy for M I {C}_{\text{?}} ) / 6 = \frac{425.62 \% + 13619.83 \% + 212.81 \% + \\ 425.62 \% + 425.62 \% + 425.62 \%}{6} = 2589.19 \%$$

$$\text{?} \text{indicates text missing or illegible when filed}$$

Then these values were averaged through MIC, MBC/MFC, and MBIC categories.

$$Average \% {Efficacy}_{\text{?}} = ( Average \% Efficacy for M I {C}_{\text{?}} + Average \% Efficacy for M B {C}_{\text{?}} + Average \% Efficacy for M B I {C}_{\text{?}} ) / 3$$

$$\text{?} \text{indicates text missing or illegible when filed}$$

Synergism Susceptibility Testing of Microbial Planktonic Growth. Synergism susceptibility testing of microbial planktonic growth was conducted using methods similar to MIC and MBC for single antibacterial agents wherein serial dilutions of the antibacterial agents individually and in combination were carried out along the rows of three microtiter plates.

Antibacterials were 2-fold diluted with MHB in each column, reaching a volume of 75 μL. The 1.0 McFarland standard was diluted 15-fold in MHB, and 75 μL of inoculum was added to the wells treated with antibacterials, resulting in a total volume of 150 μL. The microtiter plates were covered, then shake-incubated overnight at 150 rpm and 37° C. MIC was determined based on visible bacterial growth. In cases of antibacterial opacity or ambiguous results, streaking of well cultures on MHA, overnight incubation, and subsequent comparison to controls were performed. Synergistic MBC was determined by transferring 3 μL of culture from all MIC microtiter plate wells to 147 μL of MHB in fresh plates. These plates were shake-incubated overnight at 150 rpm and 37° C., and bacterial growth was visually inspected.

Different candidate antimicrobial compositions were prepared by applying components and combinations of components according to Table 1.

Results averaged to arrive at the total antimicrobial efficacy percent over silver alone.

Synergism potency of select candidate antimicrobial combinations were then evaluated according to Table 2. Table 2 provides averages a minimum of three biological repeats.

TABLE 1

| Antimicrobial | Agent # | | | | |
|---|---|---|---|---|---|
| Composition | A | B | C | D | E |
| Ratio | Zinc | Carvacrol | THC | CBD | Resveratrol |
| Molar ratio (min | 1 | 10 | 0.5 | 0.5 | 2 |
| range) | | | | | |
| Molar ratio | 1 | 1 | 0.025 | 0.025 | 0.2 |
| (max range) | | | | | |
| Molar ratio | 1 | 3 | 0.1 | 0.1 | 1 |
| (best ratio) | | | | | |

TABLE 2

| | | Total antimicrobial |
|---|---|---|
| | Antimicrobial | efficacy percent over |
| | Composition with | silver alone, including |
| | molar ratios of; | Antibacterial and |
| Number of | Zinc 1:Carvacrol 3:Resveratrol | antibiofilm efficacy |
| Components of | 1:THC 0.1:CBD 0.1 | (%) |
| 5 | Zn Carvacrol Resveratrol | 1564 |
| | THC CBD | |
| 4 | Zn Carvacrol THC CBD | 1124 |
| | Zn Carvacrol | 1045 |
| | Resveratrol THC | |
| | Zn Carvacrol | 987 |
| | Resveratrol CBD | |
| 3 | Zn Carvacrol THC | 384 |
| | Zn Carvacrol | 375 |
| | Resveratrol | |
| | Zn Carvacrol CBD | 358 |
| 2 | Zn Carvacrol | 12.9 |
| | Zn Thymol | 12.7 |
| | Zn Tea Tree | 6.0 |
| 1 | Zn | 4.7 |
| | Carvacrol | 1.3 |
| | Tea Tree | 0.5 |

### Example 2

Samples from Example 1 were tested using combinations of additives as well as additives on their own for measuring bacteriostatic effects, bactericidal effects and biofilm inhibition properties.

Method of bacteriostatic effects, bactericidal effects explained in example 1 using MIC and MBC methods, respectively.

For the prevention of biofilm formation, desired concentrations of metals (loid) and/or PBCs were added to 96 wells, with 1.0×10⁶ CFU/ml bacteria/candida added to each well. The plates were then incubated for 48 hours at 37° C. in a microplate shaker incubator at 150 rpm. Afterward, the planktonic cells and spent medium were discarded, and the adhered biomass was rinsed twice with distilled water. Biofilm biomass and minimum biofilm inhibitory concentration (MBIC) were determined using the crystal violet assay. The well with no bacterial biofilm and an OD600 absorption of zero was considered the MBIC. Results from at least three separate biological replicates were averaged.

In the crystal violet assay, the microplates or Calgary biofilm device were rinsed twice with 200 μl of 0.9% saline, then stained with 200 μl of a 0.1% crystal violet solution for 30minutes. Following staining, the microplates and pegs were washed three times with 200 μl of ddH₂O to remove excess dye. Biofilm quantification was performed by sonication using a 250HT™ ultrasonic cleaner (VWR International) set at 60 Hz for 10 minutes into 200 μl of 70% ethanol, and absorbance was read at 600 nm using 70% ethanol as the blank.

Different candidate antimicrobial compositions were prepared by applying components and combinations of components according to Table 3.

The results averaged to arrive at the bacteriostatic, bactericidal and anti-biofilm values shown in Table 3.

TABLE 3

| | | | Biofilm |
|---|---|---|---|
| | Bacteriostatic | Bactericidal | Inhibition |
| Components | (MIC) | (MBC) | (MBIC) |
| Zn 1:Carvacrol 1 | 0.25 | 0.25 | 0.25 |
| H₂O₂ (3%) | 0.25 | 0.5 | 0.5 |
| Zn (1%)-Carvacrol (1%) + | 0.0625 | 0.0625 | 0.0625 |
| H₂O₂ (3%) | | | |
| H₂O₂ (1.5%) | 0.5 | 0.5 | 0.5 |
| Zn (1%)-Carvacrol (1%) + | 0.125 | 0.125 | 0.125 |
| H₂O₂ (1.5%) | | | |
| Liquid | 0.25 | (0.25-0.5) | (0.25-0.5) |
| BZK (0.1%) | | | |
| Zn (1%)-Carvacrol (1%) + | 0.0625 | 0.0625 | 0.0625 |
| Liquid BZK (0.1%) | | | |
| Powder | 0.25 | (0.25-0.5) | (0.25-0.5) |
| BZK (0.1%) | | | |
| Zn (1%)-Carvacrol (1%) + | 0.0625 | 0.0625 | 0.0625 |
| Powder BZK (0.1%) | | | |
| Betaine (0.1%) | 1 | 1 | 1 |
| Zn (1%)-Carvacrol (1%) + | 0.25 | 0.25 | 0.125 |
| Betaine (0.1%) | | | |

### Example 3

Samples from Examples 1 and 2 were tested using combinations of additives as well as additives on their own for measuring biofilm biomass disruption properties.

A sample of S. aureus, a sample of P. aeruginosa, and a sample of a mixture of S. aureus, P. aeruginosa and C. albicans in a mass ratio of 1:1:1 were cultivated for 3 days to form a chronic biofilm.

For biofilm disruption, bacteria and/or fungi were cultivated in the Calgary biofilm device for 3 days at 37° C. in a microplate shaker incubator at 150 rpm. After incubation, planktonic cells and spent medium were discarded, and the adhered biomass was rinsed twice with distilled water. The remaining biofilm biomass was determined using the crystal violet assay as described in Example 2. Different candidate antimicrobial composition were prepared by combining various agents according to Table 4 and method of Example 1. The candidate antimicrobial compositions were substantially free of silver. In the context of the present disclosure, the term “free of silver” refers to having 0.01 mol % or less silver atoms, such as having 0 mol % silver atoms.

Table 4 shows a summary of the candidate antibiofilm compositions.

TABLE 4

| Candidate | Candidate |
|---|---|
| Antimicrobial | Antimicrobial |
| Composition | Composition |
| Formulation Number | Formulation |
| Formulation #3 | Zn 1:Carvacrol 1 |
| Formulation #11 | Formulation #3 + Thymol (0.1%) |
| Formulation #13 | Formulation #3 + Car (0.2%) |
| Formulation #14 | Formulation #3 + Zn (0.2%) |
| Formulation #15 | Formulation #3 + ZnOx (0.2%) |
| Formulation #16 | Betaine (0.2%) |
| Formulation #17 | Formulation #3 + BZK powder (0.2%) |
| Formulation #18 | Formulation #3 + BZK liquid (0.2%) |
| Formulation #19 | Formulation #3 + H₂O₂ (0.2%) |
| Formulation #21 | Formulation #3 + Coco Glucose (0.2%) |
| Formulation #22 | Formulation #3 + Tween ® 20 (0.2%) |
| Formulation #23 | Formulation #3 + SDS (0.2%) |
| Formulation #24 | Formulation #3 + CTAB (0.2%) |
| Formulation #25 | Formulation #3 + Betaine (Sigma) (0.2%) |
| Formulation #26 | Formulation #3 + Betaine |
| | (Thermo Science) (0.2%) |
| Formulation #27 | Formulation #3 + EDTA (0.2%) |
| Formulation #28 | Formulation #3 + Saponin (0.2%) |
| Formulation #29 | Formulation #3 + Sucrose fatty acid (0.2%) |
| Formulation #30 | Formulation #3 + Lauroylsarcosince (0.2%) |
| Formulation #31 | Formulation #3 + Betaine Surfactant (0.2%) |
| Formulation #32 | Formulation #3 + SCI (0.2%) |
| Formulation #33 | Formulation #3 + PHMB (0.2%) |

Biofilm disruption potency of the candidate antimicrobial compositions was measured using crystal violet assays to determine the proportion of biofilm biomass remaining following about 5 minutes and 30 minutes of contact with a candidate antimicrobial composition.

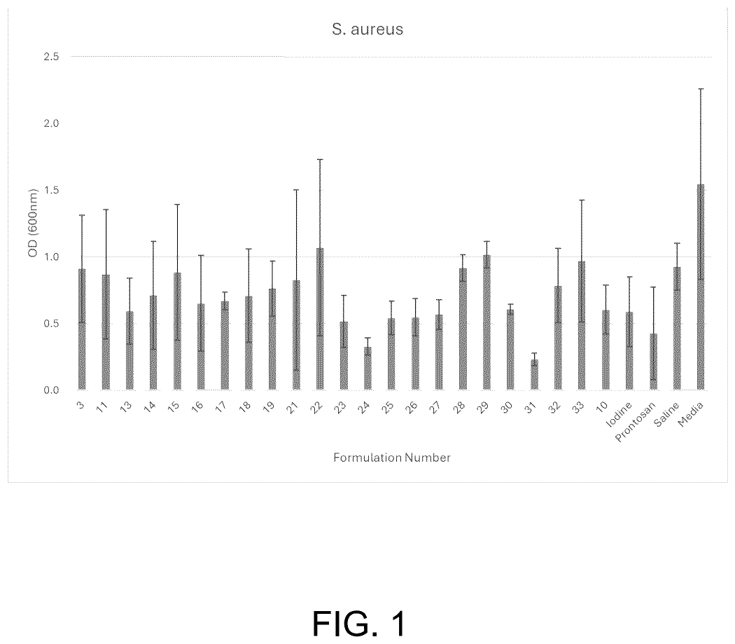

FIG. 1 is a bar graph showing the optical density (OD) at 600 nm of the S. aureus biofilm on the y-axis after about 30 minutes of contact with the different candidate antimicrobial compositions shown on the x-axis. Control candidates are also shown, including iodine, Prontosan® irrigation solution, saline (0.9%) NaCl, and growth on Simulated Wound Fluid (SWF).

A lower optical density (OD) indicates a more effective biofilm disrupting composition.

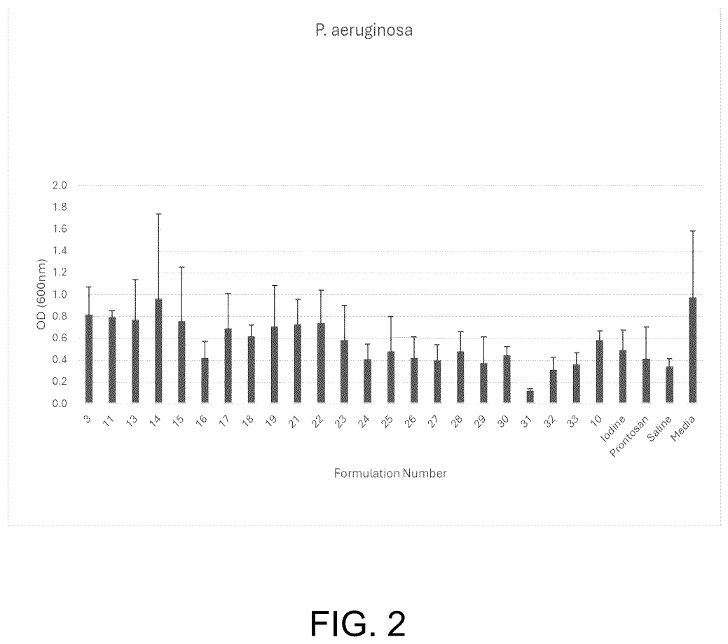

FIG. 2 is a bar graph showing the OD at 600 nm of the P. aeruginosa biofilm on the y-axis after about 30 minutes of contact with the different candidate antimicrobial compositions shown on the x-axis. Control candidates are also shown, including iodine, Prontosan® irrigation solution, saline (0.9%) NaCl, and growth on Simulated Wound Fluid (SWF).

A lower OD indicates a more effective candidate antimicrobial composition.

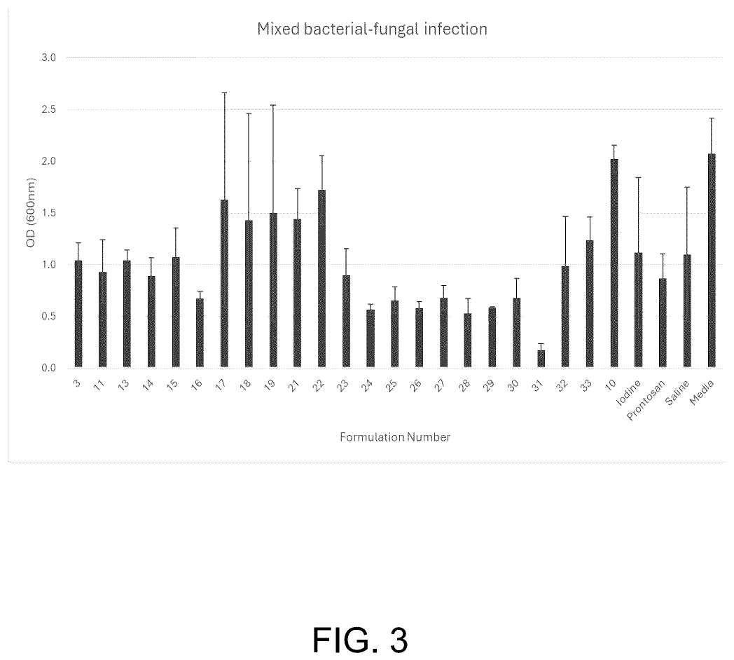

FIG. 3 is a bar graph showing the OD at 600 nm of the S. aureus, P. aeruginosa and C. albicans biofilm on the y-axis after about 30 minutes of contact with the different candidate antimicrobial compositions shown on the x-axis. Control candidates are also shown, including iodine, Prontosan® irrigation solution, saline (0.9%) NaCl, and growth on Simulated Wound Fluid (SWF).

A lower OD indicates a more effective candidate antimicrobial composition.

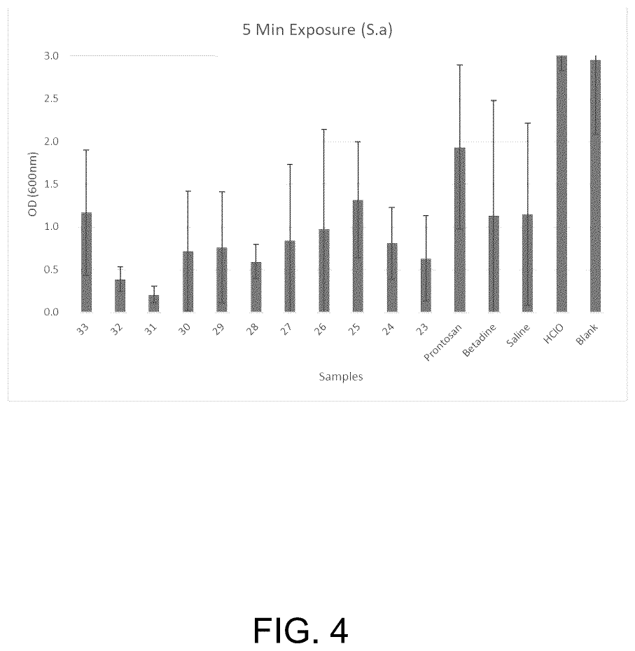

FIG. 4 is a bar graph showing the OD at 600 nm of the S. aureus biofilm on the y-axis after about 5 minutes of contact with the different candidate antimicrobial compositions shown on the x-axis. Control candidates are also shown, including iodine, Prontosan® irrigation solution, saline (0.9% NaCl), and growth on Simulated Wound Fluid (SWF).

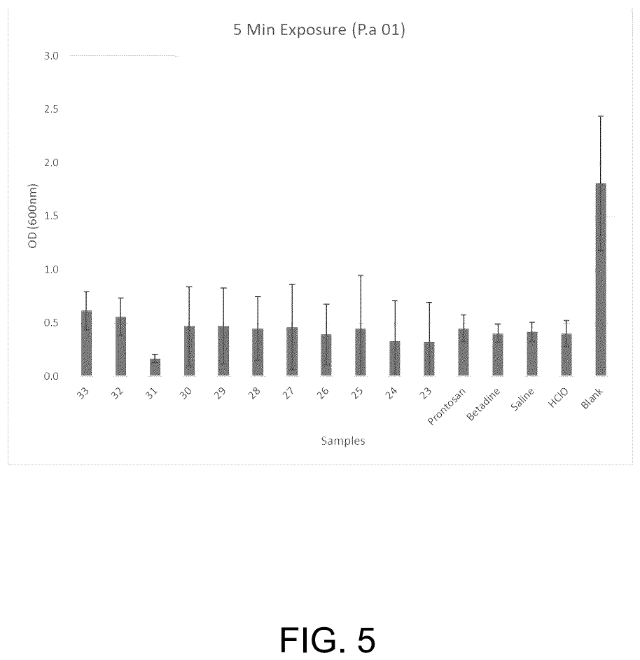

FIG. 5 is a bar graph showing the OD at 600 nm of the P. aeruginosa biofilm on the y-axis after about 5 minutes of contact with the different candidate antimicrobial compositions shown on the x-axis. Control candidates are also shown, including iodine, Prontosan® irrigation solution, saline (0.9% NaCl), and growth on Simulated Wound Fluid (SWF).

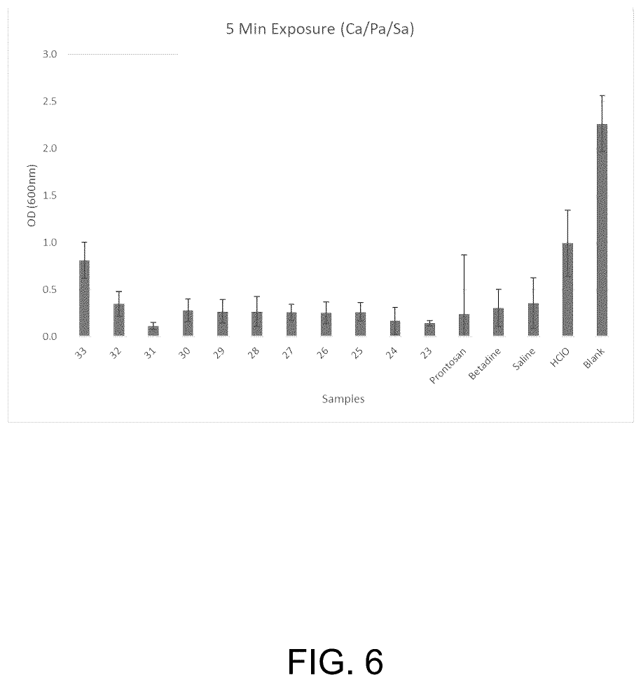

FIG. 6 is a bar graph showing the OD at 600 nm of the mixed S. aureus, P. aeruginosa and C. albicans biofilm on the y-axis after about 5 minutes of contact with the different candidate antimicrobial compositions shown on the x-axis. Control candidates are also shown, including iodine, Prontosan® irrigation solution, saline (0.9% NaCl), and growth on Simulated Wound Fluid (SWF).

### Example 4

Formulation #31 from Examples 3 was selected and applied to wound dressings to evaluate its ability to prevent the growth of microbes, compared to silver-coated wound dressings available in the market such as Cura Vance™ Alginate A—Silver Calcium Alginate Dressing and Areza Silver Alginate Dressing, against the most common bacteria and fungi responsible for wound infections.

### Deep Coating on Wound Dressings

Two coating polymers, PEG 2000 (10%) and PVA (3%), were utilized to coat the wound dressings. The deep coating method was employed, wherein the dressings were immersed in the different solutions for 10 seconds and allowed to dry for 2 hours at room temperature (24° C.). This deep coating process was repeated three times to ensure proper surface coverage, with the dressings then left to dry for 72 hours at room temperature.

To assess bandage contamination, both coated and uncoated bandages were cut to the same shape and size (22×22 mm) and placed in 12-well plates. These bandages were then soaked with Simulated Wound Fluid (SWF), and 1.0×10⁶ CFU/ml bacteria/candida was added to cach well. The plates were subsequently incubated for 24 hours at 37° C. Colony-forming units (CFU) were enumerated to determine the exact amount of bacterial growth on the contaminated bandages.

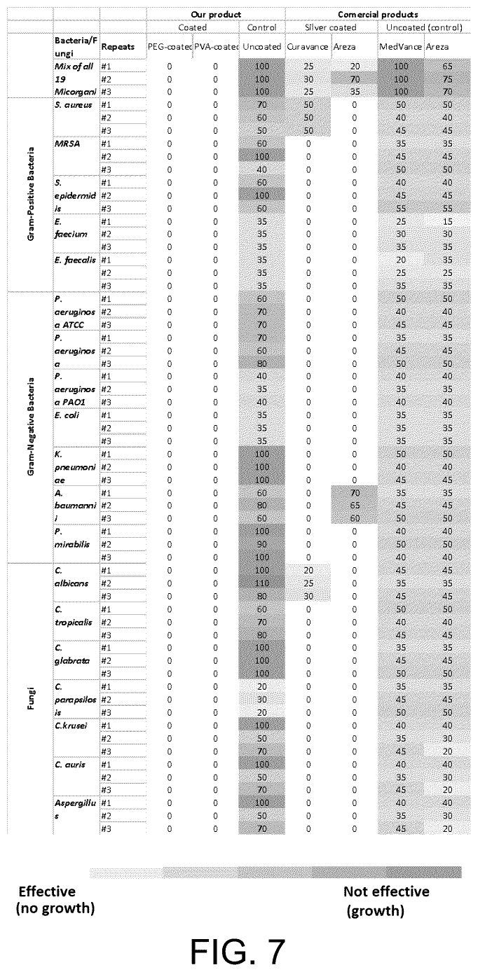

FIG. 7 depicts a heat map illustrating the antibacterial efficacy of the coated bandages with formulation #31 compared to two available silver-coated bandages across 19 indicator strains. Coated bandages may demonstrate the ability to inhibit the growth of not only single microbial strains but also mixed microbial populations.

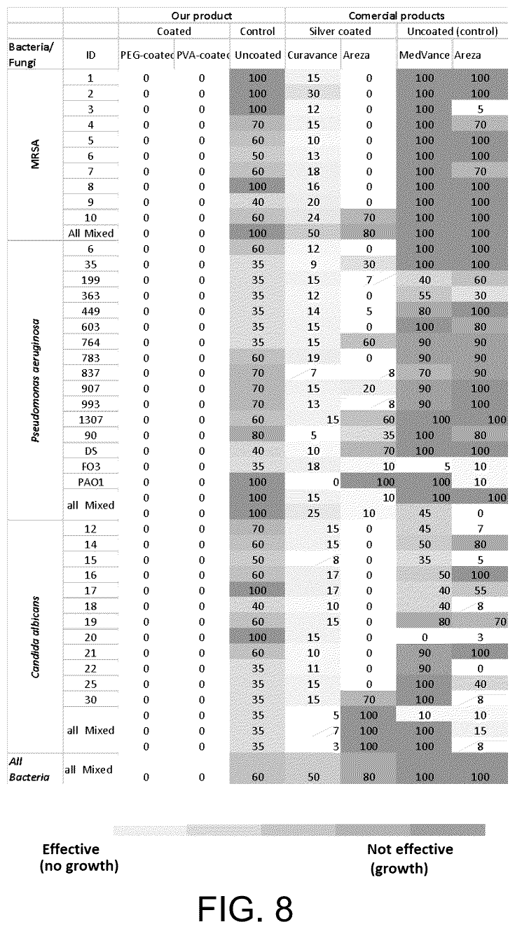

FIG. 8 illustrates a heat map showcasing the antibacterial efficacy of the coated bandages with formulation #31 compared to two available silver-coated bandages across 38 clinical isolates of bacteria and fungi. Coated bandages may demonstrate the ability to inhibit the growth of not only single clinical isolates but also mixed clinical populations.

### Example 5

Formulation #31 from Example 3 was selected for the rabbit model to see its anti-inflammatory properties. These experiments have been done by a third-party GLP-level lab.

Animal Model: This study was designed to assess the potential irritation, biological reactivity, and inflammatory properties of formulation #31 after intracutaneous injection in New Zealand White rabbits. This model was chosen because it is recommended by the ISO 10993-23 guideline for in vivo biocompatibility studies due to the lack of validated non-animal alternatives for such evaluations. New Zealand White rabbits have been widely used in biocompatibility testing for their predictable and translatable response to human clinical scenarios.

Experimental Design: Formulation #31 (test article) and Prontosan Wound Gel (control article) were sent to 3^{rd} party lab. No additional processing or dilution of the products occurred before their administration to the animals. Three New Zealand White rabbits were used in this study. Prior to the start of the experiment, cach animal underwent a pre-treatment screening to ensure they were free from irritation, trauma, or pre-existing conditions that could affect the outcome of the test. Body weights were recorded before treatment administration, and the animals' dorsal sides were clipped of fur 4-18 hours before the injections. For each rabbit, five intracutaneous injection sites were selected on either side of the dorsal region. The test article was injected at five sites on one side, while the control article was injected at five sites on the other side, with 0.2 mL of the respective products injected into each site. The injection sites were marked with indelible ink for easy identification during the post-dose observation periods.

Post-Dose Observations: The injection sites were observed immediately after administration and at intervals of 24±2 hours, 48±2 hours, and 72±2 hours. Clinical observations, including erythema (skin redness) and edema (swelling), were scored using a standard Classification System for Scoring Skin Reactions. Body weights were recorded again at the end of the observation period to monitor any changes. At the conclusion of the study, the rabbits were humanely euthanized using an injectable barbiturate. The injection sites, along with surrounding tissue, were excised and fixed in 10% neutral buffered formalin for histological evaluation.

Histopathological Evaluation: The fixed tissues were processed at Labcorp (Madison, Wisconsin). Hematoxylin and eosin-stained sections were prepared, and microscopic examination was performed to assess biological reactions, including signs of inflammation (e.g., infiltration of inflammatory cells), encapsulation, hemorrhaging, necrosis, and discoloration. Macroscopic evaluations were also performed before the histological assessment. The following categories of biological reaction were assessed by microscopic observation and the responses graded according to the following table for each injection site:

Table 5 shows microscopic observation grad for inflammatory response

TABLE 5
Inflammatory Responses

| | Score | | | | |
|---|---|---|---|---|---|
| Cell Type/Response | 0 | 1 | 2 | 3 | 4 |
| Polymorphonuclear Cells | 0 | Rare, 1-5/phf^{a} | 5-10/phf | Heavy Infiltrate | Packed |
| Lymphocytes | 0 | Rara, 1-5/phf | 6-10/phf | Heavy Infiltrate | Packed |
| Plasma Cells | 0 | Rare, 1-5/phf | 5-10/phf | Heavy Infiltrate | Packed |
| Macrophages | 0 | Rare, 1-5/phf | 5-10/phf | Heavy Infiltrate | Packed |
| Giant Cells | 0 | Rare, 1-2/phf | 3-5/phf | Heavy Infiltrate | Sheets |
| Necrosis | 0 | Minimal | Mild | Moderate | Severe |

^{a}phf = per high powered (400×) field.

Table 6 shows microscopic observation grad for healing response

TABLE 6
Healing Responses

| Cell Type/ | Score | | | | |
|---|---|---|---|---|---|
| Response | 0 | 1 | 2 | 3 | 4 |
| Neovascularization | 0 | Minimal capillary, | Groups of 4-7 | Broad band of | Extensive band |
| | | proliferation, | capillaries with | capillaries with | of capillaries with |
| | | focal, 1-3 buds. | supporting | supporting | supporting |
| | | | fibroblastic | structures | fibroblastic |
| | | | structures | | structures |
| Fibrosis | 0 | Narrow band | Moderately thick | Thick band | Extensive band |
| | | | band | | |
| Fatty Infiltrate | 0 | Minimal amount | Several layers of | Elongated and | Extensive fat |
| | | of fat associated | fat and fibrosis | broad | completely |
| | | with fibrosis | | accumulation of | surrounding the |
| | | | | fat cells about the | implant |
| | | | | implant site | |

The relative size of the involved area was scored by assessing the width of the area from the implant/tissue interface to unaffected areas which have the characteristics of normal tissue and normal vascularity. Relative size of the involved area was scored using the following scale:
0 = 0 mm, No site
1 = up to 0.5 mm, Very slight
2 = 0.6-1.0 mm, Mild
3 = 1.1-2.0 mm, Moderate
4 = >2.0 mm, Severe

After the 72±2 hours grading, all erythema grades plus edema grades from 24±2 hours, 48±2 hours, and 72±2 hours were totaled separately for each test article or vehicle control for each individual animal. To calculate the score of a test article or vehicle control on each individual animal, divide each of the totals by 15 (3 scoring time points x 5 test or vehicle control injection sites). To determine the overall mean score for each test article and each corresponding vehicle control, the scores were added for the three animals and divided by three. The final test article score was obtained by subtracting the score of the vehicle control from the test article score. The requirements of the test will be met if the difference between the test article mean score and the vehicle control mean score is 1.0 or less. If at any observation period the average reaction to the test article is questionably greater than the average reaction to the vehicle control, the test will be repeated using three additional rabbits. Each injection site was graded for inflammation and healing responses, and the scores were totaled to determine a Bioreactivity Rating. The rating was calculated by averaging the scores across all animals and comparing the test article's performance with the control article. The Bioreactivity Rating system used was as follows:
0.0-2.9: No Reaction
3.0-8.9: Slight Reaction.
9.0-15.0: Moderate Reaction
15: Severe Reaction

Data Analysis: Erythema and edema scores from each observation period were combined and averaged for each injection site. The overall mean score for the test article was calculated by subtracting the score of the vehicle control from the test article score. A Bioreactivity Rating of ≤1.0 was considered acceptable based on ISO 10993-23 standards. If the results indicated questionable reactivity, the study protocol required repeating the experiment with additional rabbits.

### Result:

Animal Weight and Clinical Observations; All animals maintained stable body weights throughout the study. No overt signs of weight loss or systemic toxicity were noted in any of the animals during the observation period.

Macroscopic Observations: Macroscopic evaluation of the injection sites indicated mild to moderate discoloration at all test treatment sites. Mild discoloration was also observed at three control sites. No significant signs of inflammation, encapsulation, hemorrhage, or necrosis were observed during the 72-hour observation period. These observations suggest minimal adverse tissue reactions for both the test and control. The sites injected with the test article did not show a significantly greater biological reaction than the sites treated with the control article (Table 7).

Table 7 shows the intracutaneous test skin reaction score

TABLE 7
Intracutaneous Test Skin Reaction Scores

| | | Site Numbers | | | | | | | | | |
|---|---|---|---|---|---|---|---|---|---|---|---|
| | | Scoring (ER/ED) | | | | | | | | | |
| Animal # | Time | T - 1 | T - 2 | T - 3 | T - 4 | T - 5 | C - 1 | C - 2 | C - 3 | C - 4 | C - 5 |
| B4491 | 0 hours† | 0/0 | 0/0 | 0/0 | 0/0 | 0/0 | 0/0 | 0/0 | 0/0 | 0/0 | 0/0 |
| | 24 hours | 1/2 | 1/2 | 1/2 | 1/2 | 1/2 | 1/2 | 1/2 | 1/2 | 1/2 | 1/2 |
| | 48 hours | 1/1 | 1/1 | 1/1 | 1/1 | 1/1 | 1/1 | 1/1 | 1/1 | 1/1 | 1/1 |
| | 72 hours | 1/1 | 1/1 | 1/1 | 1/1 | 1/1 | 1/1 | 1/1 | 1/1 | 1/1 | 1/1 |
| B4509 | 0 hours† | 0/0 | 0/0 | 0/0 | 0/0 | 0/0 | 0/0 | 0/0 | 0/0 | 0/0 | 0/0 |
| | 24 hours | 1/2 | 1/2 | 1/2 | 1/2 | 1/2 | 1/2 | 1/2 | 1/2 | 1/2 | 1/2 |
| | 48 hours | 1/1 | 1/1 | 1/1 | 1/1 | 1/1 | 1/1 | 1/1 | 1/1 | 1/1 | 1/1 |
| | 72 hours | 1/1 | 1/1 | 1/1 | 1/1 | 1/1 | 1/1 | 1/1 | 1/1 | 1/1 | 1/1 |
| B4507 | 0 hours† | 0/0 | 0/0 | 0/0 | 0/0 | 0/0 | 0/0 | 0/0 | 0/0 | 0/0 | 0/0 |
| | 24 hours | 1/2 | 1/2 | 1/2 | 1/2 | 1/2 | 1/2 | 1/2 | 1/2 | 1/2 | 1/2 |
| | 48 hours | 1/2 | 1/2 | 1/2 | 1/2 | 1/2 | 1/2 | 1/2 | 1/2 | 1/2 | 1/2 |
| | 72 hours | 1/2 | 1/2 | 1/2 | 1/2 | 1/2 | 1/2 | 1/2 | 1/2 | 1/2 | 1/2 |

Total Scores

| | |
|---|---|
| (ER + ED) | *Individual Score |

| Animal # | Test | Control | Test | Control |
|---|---|---|---|---|
| B4491 | 35 | 35 | 2.3 | 2.3 |
| B4509 | 35 | 35 | 2.3 | 2.3 |
| B4507 | 45 | 45 | 3.0 | 3.0 |
| | | ** Overall Mean | 2.6 | 2.6 |
| | | Score | | |

†= Immediately after injection not used for the evaluation criteria
*Individual Score = Total (ER + ED) divided by 15 (3 grading periods × 5 test or control sites)
** Overall Mean Score = Total Individual Scores divided by 3 animals
Overall Mean Score for Test Article = 2.6
Overall Mean Score for Control Article = 2.6
Difference between Test Article and Control Article Overall Mean Score = 2.6 − 2.6 = 0.0
ER = Erythema
T = Test Site
ED = Edema
C = Control Site

Table 8 shows ISO intracutaneous test microscopic observation.

TABLE 8
ISO Intracutaneous Test Macroscopic Observations (3 Days)

| | | | | | | | | | | |
|---|---|---|---|---|---|---|---|---|---|---|
| Tissue Site | T1 | T2 | T3 | T4 | T5 | C1 | C2 | C3 | C4 | C5 |

Animal #: B4491

| | | | | | | | | | | |
|---|---|---|---|---|---|---|---|---|---|---|
| Infection | 0 | 0 | 0 | 0 | 0 | 0 | 0 | 0 | 0 | 0 |
| Encapsulation | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 |
| Necrosis | 0 | 0 | 0 | 0 | 0 | 0 | 0 | 0 | 0 | 0 |
| Discoloration | 1 | 1 | 2 | 1 | 1 | 1 | 0 | 0 | 0 | 0 |

Animal #: B4509

| | | | | | | | | | | |
|---|---|---|---|---|---|---|---|---|---|---|
| Infection | 0 | 0 | 0 | 0 | 0 | 0 | 0 | 0 | 0 | 0 |
| Encapsulation | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 |
| Necrosis | 0 | 0 | 0 | 0 | 0 | 0 | 0 | 0 | 0 | 0 |
| Discoloration | 1 | 1 | 1 | 1 | 1 | 0 | 1 | 0 | 0 | 0 |

Animal #: B4507

| | | | | | | | | | | |
|---|---|---|---|---|---|---|---|---|---|---|
| Infection | 0 | 0 | 0 | 0 | 0 | 0 | 0 | 0 | 0 | 0 |
| Encapsulation | 0 | 0 | 0 | 0 | 0 | 0 | 0 | 0 | 0 | 0 |
| Hemorrhage | 0 | 0 | 0 | 0 | 0 | 0 | 0 | 0 | 0 | 0 |
| Necrosis | 0 | 0 | 0 | 0 | 0 | 0 | 0 | 0 | 0 | 0 |
| Discoloration | 1 | 2 | 2 | 2 | 1 | 0 | 0 | 1 | 0 | 0 |

T = Test
C = Control
Grading Scale
0 = no reaction
1 = mild reaction
2 = moderate reaction
3 = severe reaction

### Microscopic Observations

Histological analysis of the control article injection sites revealed mild edema and moderate infiltration by inflammatory cells, and multifocal small areas of connective tissue disruption were apparent within regions of inflammation. Some mononuclear cells were swollen with foamy material, without signs of necrosis or significant disruption of connective tissue. On the other hand, test article injection sites revealed significantly fewer inflammatory cells.

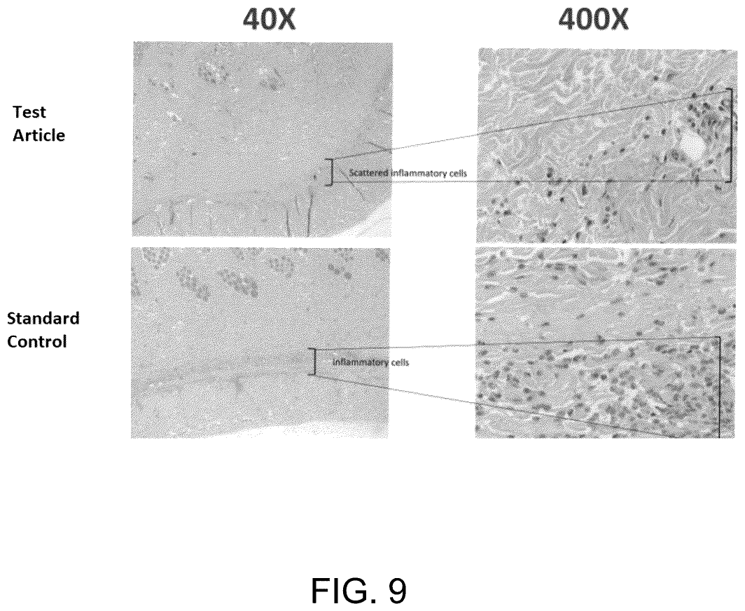

FIG. 9 illustrates histological effects such as inflammatory cells scattering of the control and test article injection sites.

### ADDITIONAL EXAMPLES

One aspect the present disclosure provides a composition comprising zinc and carvacrol in a molar ratio from about 1:about 1 to about 1:about 10, such as a ratio of about 1:about 3.

In some examples, the ratio of zinc to carvacrol may vary according to a target microbe or severity of topical infection.

In some examples, the composition may comprise delta-9-tetrahydrocannabinol (THC), wherein the zinc and the THC are present in a molar ratio from about 1:about 0.5 to about 1:about 0.025, such as a ratio of about 1:about 0.1.

In some examples, the ratio of zinc to THC may vary according to a target microbe or severity of topical infection.

In some examples, the THC may be a liquid.

In some examples, the composition may comprise cannabidiol (“CBD”), wherein the zinc and the CBD are present in a molar ratio from about 1:about 0.5 to about 1:about 0.025, such as a ratio of about 1:about 0.1.

In some examples, the ratio of zinc to CBD may vary according to a target microbe or severity of topical infection.

In some examples, the CBD may be a liquid.

In some examples, the composition may comprise comprising resveratrol, wherein the zinc and the resveratrol are present in a molar ratio from about 1:about 2 to about 1:about 0.2, such as a ratio of about 1:about 1.

In some examples, the ratio of zinc to resveratrol may vary according to a target microbe or severity of topical infection.

In some examples, the resveratrol may be a liquid.

In another aspect, the composition may be used for reducing or preventing a local infection.

In some examples, the local infection may comprise bacteria or fungi or a combination thereof.

In some examples, the local infection may comprise microbes which may be in the form of a biofilm and/or microbes in a planktonic state.

In some examples, the local infection may be a skin infection. In some examples, the local infection may be a wound infection.

In another aspect is provided method of making a composition comprising: combining zinc and carvacrol in a molar ratio from about 1:about 1 to about 1:about 10, such as a ratio of about 1:about 3.

In some examples, the method may further comprise combining zinc, carvacrol and THC, wherein the zinc and the THC are combined in a molar ratio from about 1:about 0.5 to about 1:about 0.025, such as a ratio of about 1:about 0.1.

In some examples, the ratio of zinc to THC may vary according to a target microbe or severity of topical infection.

In some examples, the method may comprise combining CBD, wherein the zinc and the CBD are combined in a molar ratio from about 1:about 0.5 to about 1:about 0.025, such as a ratio of about 1:about 0.1.

In some examples, the ratio of zinc to CBD may vary according to a target microbe or severity of topical infection.

In some examples, the method may comprise combining resveratrol, wherein the zinc and the resveratrol are combined in a molar ratio from about 1:about 2 to about 1:about 0.2,such as a ratio of about 1:about 1.

In some examples, the ratio of zinc to resveratrol may vary according to a target microbe or severity of topical infection.

In some examples, the zinc and carvacrol may exhibit an antimicrobials synergistic effect over zinc or carvacrol alone.

In some examples, the microbes may be gram-negative or gram-positive.

In some examples, the microbes may be Pseudomonas aeruginosa ATCC 27853, Pseudomonas aeruginosa PAO1, Klebsiella pneumoniae ATCC 11296, Staphylococcus aureus ATCC 25923, Methicillin resistant S. aureus (MRSA) ATCC 33591, Escherichia coli ATCC 25922, Enterococcus faecium ATCC 19434, Staphylococcus epidermidis ATCC 12228, Enterococcus faecalis ATCC 29212, Proteus mirabilis ATCC 35659, and/or Acinetobacter baumannii CDC strain AR Bank #0033.

In some examples, the microbes may be fungi.

In some examples, the microbes may be Candida albicans ATCC 90028, Candida glabrata ATCC 2001, Candida parasilosis ATCC 22019, C. krusei ATCC 14243, C. tropicalis ATCC 13803, Candida auris ATCC MYA-5002, Aspergillus fumigatus ATCC 16404.

In some examples, different isotopes, commercial brands, delivery formats, and/or structures (including nano forms) of the selected agents may have similar antimicrobial efficacy but may vary according to other chemicals in the compositions, a delivery system, pH, target application, target microbe, and/or severity of infection.

In some aspects, the disclosure relates to a composition using synergistic complement of components and related uses according to the following Embodiments, among others:

Embodiment 1. A composition comprising: zinc and carvacrol in a molar ratio from about 1:about 0.5 to about 1:about 50, such as a ratio of about 1:about 3.

Embodiment 2. The composition of Embodiment 1, further comprising THC, wherein the zinc and the THC are present in a molar ratio from about 1:about 0.5 to about 1:about 0.025, such as a ratio of about 1:about 0.1.

Embodiment 3. The composition of Embodiments 1 or 2, further comprising CBD, wherein the zinc and the CBD are present in a molar ratio from about 1:about 0.5 to about 1:about 0.025, such as a ratio of about 1:about 0.1.

Embodiment 4. The composition of any one of Embodiments 1 to 3, further comprising resveratrol, wherein the zinc and the resveratrol are present in a molar ratio from about 1:about 2 to about 1:about 0.2, such as a ratio of about 1:about 1.

Embodiment 5. The composition of Embodiment 4, wherein the zinc, carvacrol, THC, CBD and resveratrol are present in a molar ratio from about 1:about 10:about 0.5:about 0.5:about 2 to about 1:about 1:about 0.025:about 0.025:about 0.2.

Embodiment 6. The composition of any one of Embodiments 1 to 5, further comprising a carrier, a buffer, a preservative, an additive, a surfactant, a thickener, a solvent, or any combination thereof.

Embodiment 7. The composition of any one of Embodiments 1 to 6, wherein the composition is free of silver.

Embodiment 8. A composition comprising an antimicrobially synergistic combination of zinc, carvacrol, and one or more of THC, CBD, and resveratrol.

Embodiment 9. An antimicrobial composition comprising or consisting of zinc, carvacrol, THC, CBD, and resveratrol, wherein the composition is free of silver.

Embodiment 10. A composition comprising: zinc, carvacrol, THC, CBD, and resveratrol; at least one ionic or anionic surfactant, one or more preservative, and one or more antimicrobial agent.

Embodiment 11. A composition comprising: zinc, carvacrol, THC, CBD, and resveratrol, carboxymethyl cellulose (CMC), D-Panthenol, and copper peptide.

Embodiment 12. A composition comprising: zinc sulfate, carvacrol, sodium chloride, and cocamidopropyl betaine.

Embodiment 13. Use of the composition of any one of Embodiments 1 to 6 for reducing or preventing a local infection.

Embodiment 14. Use of the composition of any one of Embodiments 1 to 6 as an antimicrobial agent wherein the composition contacts a local infection.

Embodiment 15. A method of making a composition comprising: combining zinc and carvacrol in a molar ratio from about 1:about 1 to about 1:about 10, such as a ratio of about 1: about 3.

Embodiment 16. The method of Embodiment 15, further comprising combining THC, wherein the zinc and the THC are present in a molar ratio from about 1:about 0.5 to about 1: about 0.025, such as a ratio of about 1:about 0.1.

Embodiment 17. The method of Embodiment 15 or 16, further comprising combining CBD, wherein the zinc and the CBD are present in a molar ratio from about 1:about 0.5 to about 1:about 0.025, such as a ratio of about 1:about 0.1.

Embodiment 18. The method of any one of Embodiments 15 to 17, further comprising combining resveratrol, wherein the zinc and the resveratrol are present in a molar ratio from about 1:about 2 to about 1:about 0.2, such as a ratio of about 1:about 1.

In the preceding description, for purposes of explanation, numerous details are set forth to provide a thorough understanding of the examples. However, it will be apparent to one skilled in the art that these specific details are not required. Accordingly, what has been described is merely illustrative of the application of the described examples and numerous modifications and variations are possible considering the above teachings.

Since the above description provides examples, it will be appreciated that modifications and variations can be affected to the particular examples by those of skill in the art. Accordingly, the scope of the claims should not be limited by the particular examples set forth herein, but should be construed in a manner consistent with the specification as a whole.

In the preceding description, for purposes of explanation, numerous details are set forth to provide a thorough understanding of the examples. However, it will be apparent to one skilled in the art that these specific details are not required. Accordingly, what has been described is merely illustrative of the application of the described examples and numerous modifications and variations are possible considering the above teachings.

Since the above description provides examples, it will be appreciated that modifications and variations can be affected to the particular examples by those of skill in the art. Accordingly, the scope of the claims should not be limited by the particular examples set forth herein, but should be construed in a manner consistent with the specification as a whole.

What is claimed is:

1. A composition having antimicrobial effects that may be used to reduce, prevent, or eliminate infection or inflammation in animals, said composition comprising:
zinc; and
carvacrol;
wherein said zinc and carvacrol are in a molar ratio from about 1:about 0.5 to about 1:about 50.

2. The composition of claim 1 wherein said molar ratio of zinc to carcvacrol is about 1:about 3.

3. The composition of claim 1, further comprising THC; wherein the zinc and the THC are present in a molar ratio from about 1:about 0.5 to about 1:about 0.025.

4. The composition of claim 3, wherein said molar ratio of zinc to THC is about 1:about of about 1:about 0.1.

5. The composition of claim 1, further comprising CBD, wherein the zinc and the CBD are present in a molar ratio from about 1:about 0.5 to about 1:about 0.025.

6. The composition of claim 5, wherein said molar ratio of zinc to CBD is about 1:about 0.1.

7. The composition of claim 1, further comprising resveratrol, wherein the zinc and the resveratrol are present in a molar ratio from about 1:about 2 to about 1:about 0.2.

8. The composition of claim 7, wherein said molar ratio of zinc to resveratrol is about 1:about 1.

9. The composition of claim 8, wherein the zinc, carvacrol, THC, CBD and resveratrol are present in a molar ratio from about 1:about 10:about 0.5:about 0.5:about 2 to about 1:about 1:about 0.025:about 0.025:about 0.2.

10. The composition of claim 1 further comprising:
at least one of: a carrier, a buffer, a preservative, an additive, a surfactant, a thickener, or a solvent, or any combination thereof.

11. The composition of claim 1 wherein the composition is free of silver.

12. The composition of claim 1 wherein zinc is in the form of zinc sulfate and further comprising sodium chloride; and cocamidopropyl betaine.

13. The composition of claim 12 wherein the composition is free of silver.

14. A composition having antimicrobial and/or anti-inflammatory effects, said composition comprising:
zinc; carvacrol; THC; CBD; and resveratrol; and further comprising:
at least one ionic or anionic surfactant;
one or more preservative; and
one or more antimicrobial agent.

15. A composition having antimicrobial effects that may be used to reduce, prevent, or eliminate infection or inflammation in animals, said composition comprising:
zinc; carvacrol; THC; CBD; and resveratrol; and further comprising:
carboxymethyl cellulose (CMC),
D-Panthenol, and
copper peptide.

16. A method of making a composition comprising the step of combining zinc and carvacrol in a molar ratio from about 1:about 1 to about 1:about 10.

17. The method of claim 16 wherein the molar ratio of zinc to carvacrol is about 1:about 3.

18. The method of claim 16, further comprising combining THC, wherein the zinc and the THC are present in a molar ratio from about 1:about 0.5 to about 1:about 0.025.

19. The method of claim 18 wherein the molar ratio of zinc to THC is about 1:about 0.1.

20. The method of claim 16, further comprising combining CBD, wherein the zinc and the CBD are present in a molar ratio from about 1:about 0.5 to about 1:about 0.025.

21. The method of claim 20 wherein the molar ratio of zinc to CBD is about 1:about 0.1.

22. The method of claim 16, further comprising combining resveratrol, wherein the zinc and the resveratrol are present in a molar ratio from about 1:about 2 to about 1:about 0.2.

23. The method of claim 22 wherein the molar ratio of zinc to resveratrol is about 1:about 1.

24. A composition comprising an antimicrobially synergistic combination of at least two selected from the group consisting of zinc, carvacrol, THC/CBD, and resveratrol, wherein the composition includes at least one additional excipient and is free of silver.

25. Use of the composition of claim 23 for reducing or controlling inflammation.
